(19) United States
(12) Patent Application Publication
(10) Pub. No.: US 2011/0098507 A1
(43) Pub. Date: Apr. 28, 2011

(54) Asphalt Dispersers On The Basis Of Phosphonic Acids

(75) Inventors: Carsten Cohrs, Frankfurt (DE); Stefan Dilsky, Altoetting (DE); Dirk Leinweber, Kelkheim (DE); Michael Feustel, Koengernheim (DE)

(73) Assignee: CLARIANT FINANCE (BVI) LIMITED, Tortola (VG)

(21) Appl. No.: 12/747,693

(22) PCT Filed: Dec. 3, 2008
(86) PCT No.: PCT/EP2008/010209

§ 371 Date: Dec. 8, 2010

(30) Foreign Application Priority Data
Dec. 15, 2007 (DE) 102007060651.8

(51) Int. Cl. C07F 9/38; C07C 7/20

(52) U.S. Cl. 568/14; 585/3

(57) ABSTRACT

The object of the invention is the use of phosphonic acids of the formula (4),

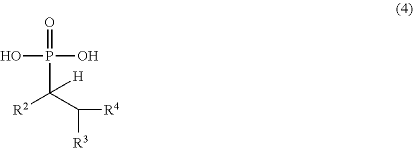

where R² is H or C₁-C₅₀₀-alkyl, and R³ and R⁴ independently from each other are H or C₁-C₅₀₀-alkyl, with the proviso that not all groups R², R³, R⁴ are hydrogen, and having a molecular weight from 250 to 10,000 units, in quantities from 0.5 to 10,000 ppm with respect to the oil, for the dispersion of asphaltenes in asphalt-containing crude oils or residual oils.

The present invention relates to the use of phosphonic acids as asphaltene dispersants in asphaltene-containing crude oils, residual oils and distillate oils.

Asphaltenes are constituents of crude oils. They contain a large number of structures, particularly high molecular weight condensed aromatic components with heteroatoms. In view of the complexity of their chemistry, asphaltenes are described as the oil fraction which is soluble in benzene, but not in pentane.

In crude oil, asphaltenes are normally in the form of a colloidal dispersion. This is stabilized by oil resins.

Asphaltenes can precipitate out during production, refining, transportation and storage of crude oil and products derived therefrom, such as, for example, heavy fuel oil or marine oil. Common causes for this precipitation are a drop in the temperature or a change in the composition (e.g. evaporation of readily volatile constituents). Asphaltenes can also precipitate out upon flowing through porous media. Flooding with CO₂ during the recovery process can cause asphaltenes to flocculate or to precipitate out.

Some oils comprise hydrocarbon waxes which precipitate out at low temperatures. Interactions between the precipitation of wax and asphaltenes can increase the overall amount of precipitated substance or its rate of formation.

Precipitated asphaltenes cause problems during the production and processing of crude oils. Asphaltenes settle out in valves, pipes and conveyors. On hot surfaces, such as, for example, heat exchangers, the carbonization of these precipitates can make their removal very difficult. The precipitates reduce the efficiency of plants and can, in the worst case, lead to complete blockage and to a halt in production, which results in high costs.

Heavy oils, which are often used for powering ships, comprise considerable amounts of asphaltenes. The precipitation of asphaltenes can lead both to poor combustion and also to difficulties with regard to handling and storage of the fuel.

Bitumens, heavy oils and residues are sometimes diluted with solvents in order to reduce the viscosity for transportation. If asphaltenes precipitate out, then problems arise during handling.

The precipitation of asphaltenes can be prevented or reduced by small amounts of dispersants. These substances exhibit one or more of the following effects:

a) the amount of precipitate is reduced
b) the precipitate is formed more slowly
c) the precipitate is more finely divided
d) the tendency of the precipitate to deposit on surfaces is reduced.

If precipitates of asphaltenes have already formed, they can be removed through the use of solvents. The addition of a dispersant can improve the effectiveness of these solvents.

A large number of asphaltene dispersants are already known. CA-A-2 029 465 and CA-A-2 075 749 describe alkylphenol formaldehyde resins in combination with hydrophilic-lipophilic vinylpolymers.

The asphaltene-dispersing properties of dodecylbenzenesulfonic acid are described in U.S. Pat. No. 4,414,035, D.-L. Chang and H. S. Fogler (SPE 25185, 1993), and by M. N. Bouts et al. (J. Pet. Technol. 47, 782-787, 1995).

A. Stiles et al., J. Am. Chem. Soc. 1958, 80, 714-716 discloses the free-radical addition of phosphonic acid diesters onto olefins.

Houben-Weyl, volume XII/1, 1963, pages 352-353, volume E2, 1982, pp. 310-311 and pp. 350-351 disclose the hydrolysis of alkylated phosphonic acid esters, the thermal cleavage of alkylated phosphonic acid esters and the reaction of olefins with phosphorous acid.

GB-A-2423099 discloses a process for avoiding the formation of crosslinking products of asphaltenes which are formed in an acidic medium in the presence of Fe(III) ions.

DE-A-10 2005 045133 discloses the use of alkylphosphonic acid esters as co-additive for asphaltene dispersants which comprise alkylphenol-aldehyde resins.

EP-1 362 087 discloses the use of cardanol-aldehyde resins as asphaltene dispersants in crude oils.

U.S. Pat. No. 5,494,607 discloses the use of nonylphenol-pentadecylphenol-formaldehyde resins as asphaltene dispersants in crude oils, the pentadecylphenol being obtained from cashew nuts.

EP-0 995 012 discloses the use of ether carboxylic acids as asphaltene dispersants in crude oils.

Phosphonic acids of the formula (4) given below with a short alkyl radical are disclosed in DE-A-199 27 787 as flame retardants and precursors in chemical synthesis. Their use as asphaltene dispersants is not described.

The synthesis of phosphonic acids of the formula (4) given below is disclosed in DE-103 05 623 starting from phosphorus halides. A halogen-free preparation process is not described.

The dispersants known to date can only partly solve the problems caused by the precipitation of asphaltenes. Since oils vary in their composition, individual dispersants can only operate effectively within a limited range, meaning that small changes in the oil composition have a great effect on the dispersing properties for asphaltenes. For this reason, in some cases the known dispersants are unsatisfactory and additional types are required. Moreover, the known dispersants can be detected only inadequately in crude oils following a “squeeze treatment”. Squeeze treatment is understood as meaning the introduction of a solution or emulsion with the application of pressure in a range in which the solution/emulsion is supposed to develop its effectiveness. In oil fields, the pressure injection of the solution/emulsion takes place here in most cases below the intrinsic pressure present in the formation.

It was therefore the object to provide novel asphaltene dispersants which do not have the described disadvantages of the dispersants known hitherto. They should be able to disperse the asphaltenes present in crude oils and residual oils to an adequate extent. Moreover, the object was to provide a process by which suitable asphaltene dispersants can be prepared, in particular long-chain-substituted phosphonic acids.

Surprisingly, it has been found that substituted phosphonic acids can be used in order to prevent the precipitation and/or the deposition of asphaltenes in crude oils and products derived therefrom. The phosphonic acids can be detected analytically by virtue of the phosphorus atom using various methods. The method used depends on the conditions in the oil field and the desired detection sensitivity. Thus, for example, ³¹P NMR spectroscopy (detection limit ca. 0.5%), inductive coupled plasma (ICP, detection limit in the ppm range), or GC flame photometer (detection limit in the ppm range) can be used for the detection.

The invention therefore provides the use of phosphonic acids of the formula (4)

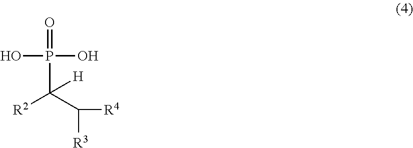

in which
R² is H or C₁-C₅₀₀-alkyl, and
R³ and R⁴, independently of one another, are H or C₁-C₅₀₀-alkyl,
   with the proviso that not all of the radicals R², R³, R⁴ are hydrogen, and which have a molecular weight of from 250 to 10 000 units, in amounts of from 0.5 to 10 000 ppm, based on the oil, for dispersing asphaltenes in asphaltene-containing crude oils or residual oils.

The invention further provides a process for the preparation of phosphonic acids of the formula (4) by reacting phosphonic acid diesters of the formula (1)

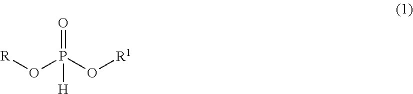

with a compound of the formula (2)

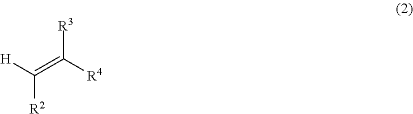

and then hydrolyzing the phosphonic acid diester of the formula (3) formed from (1) and (2)

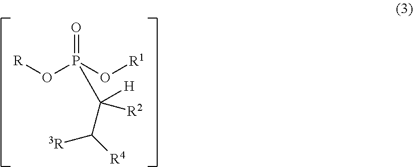

with water to give the compound of the formula (4)

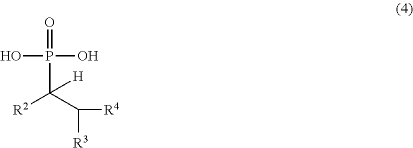

and in which
R and R¹, independently of one another, are C₁-C₁₂-alkyl, C₆-C₁₂-aryl or C₇-C₁₆-alkylaryl,
R² is H or an alkyl group having 30 to 500 carbon atoms and
R³ and R⁴, independently of one another, are H or an alkyl group having 30 to 500 carbon atoms,
   with the proviso that not all of the radicals R², R³, R⁴ are hydrogen.

This corresponds to the reaction equation

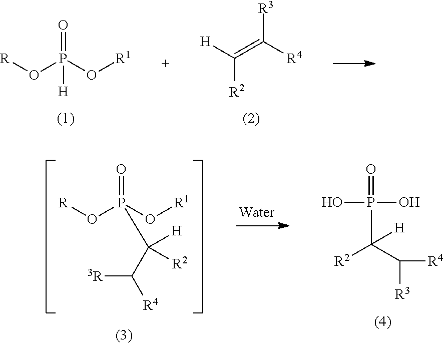

If R, R¹, R², R³ or R⁴ are alkyl groups, then these may be linear or branched.

The invention further provides asphaltene-containing crude oils or residual oils comprising 0.5 to 10 000 ppm of a compound of the formula (4) in which
R² is H or an alkyl group having 30 to 500 carbon atoms,
R³, R⁴, independently of one another, are H or an alkyl group having 30 to 500 carbon atoms,
with the proviso that not all of the radicals R², R³, R⁴ are H, and which has a molecular weight of from 250 to 10 000 g/mol.

R is preferably C₂- to C₈-alkyl radicals.

R¹ is preferably C₂- to C₈-alkyl radicals.

The radicals R², R³ and R⁴, independently of one another, are preferably long-chain radicals which comprise at least 30, in particular at least 40 and, for example, at least 50 carbon atoms. They comprise preferably at most 400, specifically 250 carbon atoms.

It is preferred that the compound of the formula (2) is a C₃₀₊-α-olefin. Preferably, suitable C₃₀₊-α-olefins have molar masses in the range from 450 to 10 000, specifically 600 to 5000 g/mol.

In a further preferred embodiment of the invention, R² is hydrogen and R³ and R⁴, independently of one another are C₁-C₅₀₀-alkyl.

In a further preferred embodiment of the invention, R² is hydrogen, R³ is hydrogen and R⁴ is C₁-C₅₀₀-alkyl.

Unless stated otherwise, all data in % or ppm refer to percentages by weight or ppm by weight.

Phosphonic acid diesters of the formula (3) are prepared by free-radical insertion of an alkene into the P—H bond of the phosphonic acid diester of the formula (1). The free-radical initiators used here are preferably peroxides, such as di-tert-butyl peroxide, or azo compounds, such as azoisobutyronitrile, in an amount of preferably 2 to 5% by weight, based on the weight of the reaction mixture. The alkenes used are preferably long-chain alkenes, such as, for example, C₃₀₊-α-olefin or polyisobutylene. The reaction temperature for the free-radical insertion is generally between 80 and 200° C., preferably between 120 and 160° C. The molar ratio between phosphonic acid ester and olefin is preferably between 0.9:1 and 1.1:1, in particular equimolar.

For the preparation of the phosphonic acids of the formula (4) according to the invention, the phosphonic acid ester of the formula (3) is saponified with a five- to twenty-fold excess of water, preferably an eight to twelve-fold excess of water with acidic or basic catalysis. Preference is given here to the acidic saponification using, for example, sulfonic acids or mineral acids such as HCl, which are used in amounts of from 0.1 to 5% by weight, based on the weight of the reaction mixture. The alcohol released during the saponification and also excess water are expediently removed azeotropically. The temperature for the saponification reaction is generally between 80 and 300° C., preferably between 120° C. and 250° C.

Compared with the process in DE-103 05 623, the preparation process according to the invention is ecologically and economically advantageous since the synthesis of the phosphonic acids of the formula (4) according to the invention takes place in a halogen-free manner.

The phosphonic acids of the formula (4) according to the invention can be used on their own or in combination with other known asphaltene dispersants. In general, enough of the phosphonic acid according to the invention is added to ensure adequate dispersion under the stated conditions. The phosphonic acids of the formula (4) according to the invention are particularly suitable as asphaltene dispersants in the so-called “squeeze treatment”, since they can be detected by various methods.

The compounds of the formula (4) can be used as asphaltene dispersant in crude oils. They are likewise suitable for residual oils which contain asphaltenes. These may be, for example, bunker oils.

### EXAMPLES

### Free-Radical Addition

### Example 1

### Conversion of di(2-ethylhexyl)phosphite with Glissopal® 1000 (BASF AG, Polyisobutylene with an Average Molecular Weight of 1000 g/mol)

46 g of di(2-ethylhexyl)phosphite (M=306) and 150 g of Glissopal® 1000 (M=1000) were initially introduced into a 500 ml four-necked flask fitted with contact thermometer, stirrer and dropping funnel. With stirring and nitrogen blanketing, the mixture was heated to 150° C. and, at this temperature, 4.9 g of di-tert-butyl peroxide (2.5%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧98%.

### Example 2

### Conversion of di(2-ethylhexyl)phosphite with Glissopal® 2300 (BASF AG, Polyisobutylene with an Average Molecular Weight of 2300 g/mol)

20 g of di(2-ethylhexyl)phosphite (M=306) and 150 g Glissopal® 2300 (M=2300) were initially introduced into a 500 ml four-necked flask fitted with contact thermometer, stirrer and dropping funnel. With stirring and nitrogen blanketing, the mixture was heated to 150° C. and, at this temperature, 4.3 g of di-tert-butyl peroxide (2.5%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧96%.

### Example 3

### Conversion of di(2-ethylhexyl)phosphite with C₃₀₊-α-olefin

27.5 g of di(2-ethylhexyl)phosphite (M=306) and 73 g of C₃₀₊-α-olefin (M=813) were initially introduced into a 250 ml four-necked flask fitted with contact thermometer, stirrer and dropping funnel. With stirring and nitrogen blanketing, the mixture was heated to 150° C. and, at this temperature, 2.5 g of di-tert-butyl peroxide (2.5%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧98%.

### Example 4

### Conversion of di(2-ethylhexyl)phosphite with Indopol® L8 (INEOS, Low Molecular Weight Polybutene)

94.4 g of di(2-ethylhexyl)phosphite (M=306) and 96 g of Indopol® L8 (M=320 g/mol) were initially introduced into a 500 ml four-necked flask fitted with contact thermometer, stirrer and dropping funnel. With stirring and nitrogen blanketing, the mixture was heated to 150° C. and, at this temperature, 9 g of di-tert-butyl peroxide (4.6%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧94%.

### Example 5

### Conversion of di(2-ethylhexyl)phosphite with Indopol® H100 (INEOS, High Molecular Weight Polybutene)

56.6 g of di(2-ethylhexyl)phosphite (M=306) and 163.8 g of Indopol® H100 (M=910 g/mol) were initially introduced into a 500 ml four-necked flask fitted with contact thermometer, stirrer and dropping funnel. With stirring and nitrogen blanketing, the mixture was heated to 150° C. and, at this temperature, 10.4 g of di-tert-butyl peroxide (4.6%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧98%.

### Example 6

### Conversion of Diethyl Phosphite with Glissopal® 1000

13 g of diethyl phosphite (M=138) and 90 g of Glissopal® 1000 (M=1000) were initially introduced into a 250 ml four-necked flask fitted with contact thermometer, stirrer and dropping funnel. With stirring and nitrogen blanketing, the mixture was heated to 150° C. and, at this temperature, 3.8 g of di-tert-butyl peroxide (3.7%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧98%.

### Example 7

### Conversion of Diethyl Phosphite with Indopol® H100

36 g of diethyl phosphite (M=138) and 227.5 g of Indopol® H100 (M=910) were initially introduced into a 500 ml four-necked flask fitted with contact thermometer, stirrer and dropping funnel. With stirring and nitrogen blanketing, the mixture was heated to 150° C. and, at this temperature, 13 g of di-tert-butyl peroxide (4.8%) were added dropwise over a period of 6 hours. When the addition was complete, the mixture was afterreacted for 4 hours at 150° C. The product was analyzed by means of ³¹P-NMR spectroscopy and the yield of phosphonic acid ester was determined as ≧85%.

### Saponification

### Example 8

### Saponification of di(2-ethylhexyl)phosphite-Glissopal® 1000 Adduct

67 g of di(2-ethylhexyl)phosphite-Glissopal® 1000 adduct (M=1314) and 1.4 g of alkylbenzenesulfonic acid (2.5%) were initially introduced into a 250 ml four-necked flask fitted with stirrer, dropping funnel and water separator. With stirring and nitrogen blanketing, the mixture was heated to 190° C. and, at this temperature, the ten-fold amount of theoretically required demineralized water (18.4 g) was slowly added dropwise. The resulting 2-ethylhexanol and also excess water was removed at 190° C. by means of the separator. The product was analyzed by means of ³¹P-NMR spectroscopy and acid number and a quantitative saponification of the phosphonic acid ester used was detected.

### Example 9

### Saponification of Diethyl Phosphite-Glissopal® 1000 Adduct

62.2 g of diethyl phosphite-Glissopal® 1000 adduct (M=1137) and 2 g of alkylbenzenesulfonic acid (2.5%) were initially introduced into a 250 ml four-necked flask fitted with stirrer, dropping funnel and water separator. With stirring and nitrogen blanketing, the mixture was heated to 150° C. and, at this temperature, the ten-fold amount of theoretically required demineralized water (20 g) was slowly added dropwise. The ethanol which formed and also excess water was removed at 150° C. by means of the separator. The product was analyzed by means of ³¹P-NMR spectroscopy and acid number and a quantitative saponification of the phosphonic acid ester used was detected.

### Example 10

### Saponification of di(2-ethylhexyl)phosphite-Indopol® H100 Adduct

60 g of di(2-ethylhexyl)phosphite-Indopol® H100 adduct (M=1225) and 1.3 g of alkylbenzenesulfonic acid (2.5%) were initially introduced into a 250 ml four-necked flask fitted with stirrer, dropping funnel and water separator. With stirring and nitrogen blanketing, the mixture was heated to 190° C. and, at this temperature, the ten-fold amount of theoretically required demineralized water (18 g) was slowly added dropwise. The 2-ethylhexanol which formed and also excess water was removed at 190° C. via the separator. The product was analyzed by means of ³¹P-NMR spectroscopy and acid number and a quantitative saponification of the phosphonic acid ester used was detected.

### Example 11

### Saponification of Diethyl Phosphite-Indopol® H100 Adduct

123 g of diethyl phosphite-Indopol® H100 adduct (M=1048) and 3 g of alkylbenzenesulfonic acid (2.5%) were initially introduced into a 500 ml four-necked flask fitted with stirrer, dropping funnel and water separator. With stirring and nitrogen blanketing, the mixture was heated to 150° C. and, at this temperature, the ten-fold amount of theoretically required demineralized water (42 g) was slowly added dropwise. The ethanol which formed and also excess water was removed at 150° C. by means of the separator. The product was analyzed by means of ³¹P-NMR spectroscopy and acid number and a quantitative saponification of the phosphonic acid ester used was detected.

### Testing and Effectiveness of Asphaltene Dispersants

### Principle of the Dispersion Test

Dispersion and precipitation of asphaltenes depend on the nature of the hydrocarbon medium. Asphaltenes are soluble in aromatic hydrocarbons, but not in aliphatic hydrocarbons. It is thus possible to test dispersants by dissolving the oil or extracted asphaltenes in an aromatic solvent and then adding an aliphatic hydrocarbon in order to produce a precipitate. Since asphaltenes are darker in color, the amount of precipitate can be determined by means of a colorimetric measurement of the supernatant liquid. The darker the supernatant liquid, the more asphaltenes remain dispersed, i.e. the better the dispersant. This test is described in CA-A-2 029 465. In the present version of the test, the precipitation medium is selected so that the asphaltenes precipitate out for the most part, but not completely. The dispersion test is carried out according to steps a) to f):
a) A 25% strength by weight solution of the oil in toluene is filtered in order to remove impurities.
b) Initially introduce 9.5 mol of heptane as precipitating agent for asphaltenes and 0.5 ml of toluene/dispersant mixture (25:1) into a small graduated glass tube which easily holds 10 ml and shake well. This corresponds to a dispersant concentration of 2000 ppm. The amount of dispersant can be varied as required. Pure toluene is used for the blank sample.
c) 0.1 ml of the filtered oil solution is then added to the small glass tube and likewise shaken well.
d) Leave the sample to stand for 2 hours without disruption in order that the precipitated asphaltenes can collect at the bottom of the tube.
e) After this time has elapsed, the volume of the sediment is estimated using the graduations, the appearance of the overall sample is recorded and 1 ml is carefully taken up using a pipette from the supernatant phase.
f) The amount pipetted off is dissolved in 5 ml of a 99:1 toluene/triethanolamine mixture and measured photometrically at 600 nm.

### Evaluation of the Dispersion Test

The dispersion A) is calculated using the following equation:

A=100(D−D₀)/D₀,

where D and D₀ are the optical density of measurement solution and blank sample. The maximum values of A, Aₘₐₓ, correspond to complete dispersion of the asphaltenes. It can be estimated by carrying out an experiment without dispersant, with toluene instead of heptane—as a result the asphaltenes remain completely dispersed. The volume of the sediment gives further information regarding the effectiveness of the dispersant. The smaller the amount of sediment, the better dispersed is the substance.

### Dispersion Effect of the Example Compounds

Using an asphaltene-rich oil, substances according to the invention and those of the prior art were tested using the dispersion test. The dispersant dose in all cases was 100 ppm.

| | Dispersion effect A [%] |
|---|---|
| Product from example 8 | 94 |
| Product from example 9 | 93 |
| Product from example 10 | 92 |
| Product from example 11 | 94 |
| Commercial product A | 88 |
| Commercial product B | 86 |
| Without dispersant | 0 |

In this experimental series, the maximum dispersion effect Aₘₐₓ was 94%.

1. A process for dispersing asphaltenes in an asphaltene-containing crude oil, residual oil or distillate oil, comprising the step of adding at least one phosphonic acid of the formula (4) 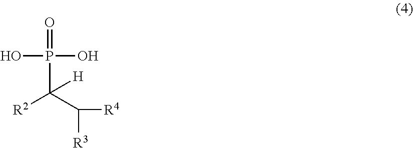
wherein
R² is H or C₁-C₅₀₀-alkyl, and
R³ and R⁴, independently of one another, are H or C₁-C₅₀₀-alkyl, with the proviso that not all of the radicals R², R³, R⁴ are hydrogen, and have a molecular weight of from 250 to 10 000 units,
to the asphaltene-containing crude oil, residual oil or distillate oil in an amount of from 0.5 to 10 000 ppm, based on the asphaltene-containing crude oil, residual oil or distillate oil.

2. A process as claimed in claim 1, where R² is hydrogen or a C₁- to C₄-alkyl radical.

3. A process as claimed in claim 1, where R² is hydrogen and R³ and R⁴, independently of one another, are C₁-C₅₀₀-alkyl.

4. A process as claimed in claim 1, wherein R² is hydrogen, R³ is hydrogen and R⁴ is C₁-C₅₀₀-alkyl.

5. A process for the preparation of a phosphonic acid of the formula (4), comprising the steps of reacting at least one phosphonic acid diester of the formula (1) 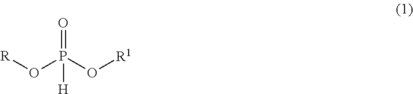
in a molar ratio from 0.9:1 to 1.1:1 with at least one compound of the formula (2) 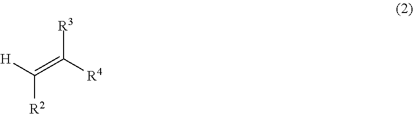
to form a phosphonic acid diester of the formula (3) and then hydrolyzing the phosphonic acid diester of the formula (3) formed from (1) and (2) 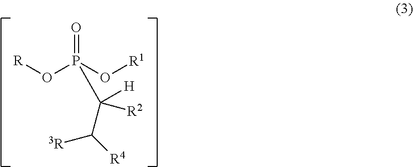
with water to form the compound of the formula (4) 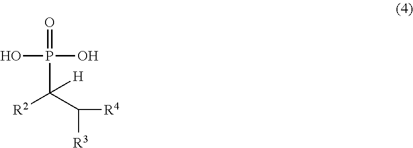
wherein
R and R¹, independently of one another, are C₁-C₁₂-alkyl, C₆-C₁₂-aryl or C₇-C₁₅-alkylaryl,
R² is H or an alkyl group having 30 to 500 carbon atoms and
R³ and R⁴, independently of one another, are H or an alkyl group having 30 to 500 carbon atoms, with the proviso that not all of the radicals R², R³, R⁴ are hydrogen.

6. A process as claimed in claim 5, wherein R is C₂- to C₈-alkyl.

7. A process as claimed in claim 5, in wherein R¹ is C₂- to C₈-alkyl.

8. (canceled)

9. A process as claimed in claim 1, in which the asphaltene dispersion takes place in the squeeze treatment.

10. A process as claimed in claim 1, further comprising the step of adding an alkylphenol formaldehyde resin, oxalkylated amine, mono- or dialkylbenzenesulfonic acid, petroleumsulfonic acid, alkanesulfonic acid, wax dispersant or any desired mixture thereof.

11. An asphaltene-containing crude oil, residual oil or distillate oil comprising 0.5 to 10 000 ppm of a compound of the formula (4) 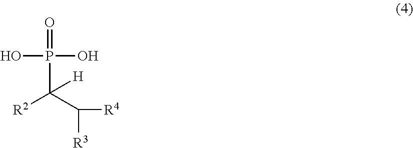
wherein
R² is H or an alkyl group having 30 to 500 carbon atoms,
R³, R⁴, independently of one another, are H or an alkyl group having 30 to 500 carbon atoms, with the proviso that not all of the radicals R², R³, R⁴ are H, and which has a molecular weight of from 250 to 10 000 g/mol.
